(19) United States
(12) Patent Application Publication
(10) Pub. No.: US 2003/0129617 A1
(43) Pub. Date: Jul. 10, 2003

(54) Calculating a biological characteristic property of a molecule by correlation analysis

(76) Inventors: Artem Tcherkassov, Vancouver (CA); Ridong Chen, Naperville, IL (US)

Correspondence Address: Philip D. Reilly, Morrison & Foerster LLP, 755 Page Mill Road, Palo Alto, CA, 94304-1018, US

(21) Appl. No.: 10/208,080

(22) Filed: Jul. 29, 2002

Related U.S. Application Data
(60) Provisional application No. 60/308,666, filed on Jul. 31, 2001.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

Methods, including computer implemented methods for calculating a biological characteristic property of a molecule from the 3D-structure of the molecule by correlation analysis, in which the contribution to the biological characteristic property from substituent parts of the molecule is equal to a function of the distance of the substituent part to a reaction center multiplied by a weight factor and substantially the same functional form of the distance function is used for calculating the contribution of each substituent part.

### CROSS-REFERENCE TO RELATED APPLICATIONS

This application claims the benefit of U.S. provisional application No. 60/308,666, filed Jul. 31, 2001, with inventors Artem Tcherkassov and Ridong Chen, which application is incorporated herein by reference. This application is related to an application filed on the same date, with the same inventors, titled, “Calculating a Characteristic Property of a Molecule By Correlation Analysis,” with attorney docket number 53260-20002.00, which application is incorporated herein by reference.

### BACKGROUND

The elucidation of the relationships between structure and activity of molecules is one of the major challenges in the chemical and pharmaceutical sciences. One approach to this problem is to apply quantitative structure—activity relationships (“QSAR”), which is a rapidly growing area, integrating methods of modern chemistry, biochemistry, pharmacology, molecular modeling, proteomics, and bio- and cheminformatics. In QSAR modeling, the activity of a molecule is estimated using the substituent parts of the molecule and the observed activity of molecules with similar or analogous structural motifs.

Application of conventional methods of QSAR have allowed interpretation of reactivity and bioactivity data and physicochemical properties of molecules. Correlation analysis, which in part is based on the principles of linearity of free energy relationships (“LFER”), is one method that has proved fruitful in this approach. Conventional correlation analysis is described in, for example, Hansch, C.; et al. Substituents Constants for Correlation Analysis in Chemistry and Biology; Wiley-Interscience: N.Y., 1979; Wells, P. R. Linear Free Energy Relationships; Academic Press: London, 1968; Chapman, N. B., Shorter, J. Correlation Analysis in Chemistry; Plenum Press, N.Y. 1978; and R. W. Parr, et al. Density-functional theory of atoms and molecules. Oxford University Press, N.Y., 1989.

Conventional correlation analysis calculates the activity of a molecule as the sum of contributions from different atoms or groups of atoms in a molecule but does not take account of the 3D-structure of the molecule and separates the contributions from each atom or group of atoms into polar, steric, inductive and resonance effects.

Quantitative description of polar influence of substituents first became possible within the framework of the approach developed by Hammett on the basis of the dissociation constants of substituted benzoic acids. The difference between the logarithms of dissociation constant K of substituted benzoic acid and the corresponding K⁰ of unsubstituted standard compound has been expressed by empirical equation:

$$log \frac{K}{{K}^{0}} = ρ σ \qquad ( 1 )$$

in which two new quantities have been introduced: σ is universal constant specific for a substituent in the benzene ring and ρ is reaction series constant reflecting the sensitivity of the reaction center to variation of substituent influence.

Later, the Hammett equation was modified many times, but the vast majority of these modifications related to the chemistry of aromatic compounds. For the series of aliphatic compounds, the Hammett relation, as a rule, did not hold. Taft suggested that in this case the steric substituent effects are significant and should be separated as:

$$log \frac{K}{{K}^{0}} = ρ {\sum }_{i} {σ}^{*} + δ {\sum }_{i} {E}_{s} \qquad ( 2 )$$

where σ* is a substituent constant depending only on the inductive influence of the substituent, E_{S} is the substituent constant reflecting the steric effect of the substituent and δ is a reaction series constant reflecting the sensitivity of the reaction center to variations of substituent steric influence. Taft's inductive and steric constants are among the most reliable and widespread substituent parameters used in conventional QSAR.

A large number of polar and steric substituent constants have been determined, and these constants are used in many different QSAR schemes that are used for analysis of molecular reactivity, bioactivity, and physicochemical properties and reaction mechanisms studies.

In terms of mechanism of action, the steric effect is believed to be due to a variety of factors including an increase of the bulk of a substituent leading to the mechanical shielding of the reaction center from an attacking reagent (steric hindrance of motions), an increase of steric repulsion in a transition state (steric strain) of a reaction, and to steric inhibition of salvation. Thus, the methods of calculation of substituents steric constants usually operate by different descriptors of effective atomic, group or molecular sizes. For the inductive effect there is no unanimously opinion as to the mechanism of action. The inductive effect includes polar electrostatic interactions between charged parts (atoms) of a molecule and polarization of bonds. The resonance effect is attributed to stabilization of a system (molecule, transition state, etc) occurring due to the realization of multiple electronic states (resonance configurations).

Although conventional QSAR methods have proved useful in elucidating structure activity relationships and predicting the activity of molecules based on their structural motifs, conventional QSAR relies on an ad hoc mixture of contributions from polar, inductive, steric and resonance effects, each of which may be treated in a different manner depending on the application. In addition, conventional QSAR does not fully take into account the three dimensional structure of a molecule and thus may not include useful and important structural information contributing to the activity of a molecule.

### SUMMARY

The inventors have identified new methods that treat the contributions from substituent parts of a molecule in a straightforward, consistent matter and take into account the full 3-D structure of a molecule when calculating the activity.

In this patent, we describe various methods that may be used to calculate the activity of a molecule based on its 3-D structure and give examples of the application of these methods demonstrating the utility of the methods. In this section, we summarize various aspects of the methods described in this patent and below in the Detailed Description section we present a more comprehensive description of these methods, their uses and implementation.

One of the methods described in this patent is a method for calculating a biological characteristic property of a molecule that includes one or more substituent parts, where the method includes the steps of (i) selecting one or more of the substituent parts as contributing substituent parts; (ii) for each of the contributing substituent parts, calculating the distance from the substituent part to a reaction center; (iii) for each of the contributing substituent parts, calculating the contribution of that substituent part to the biological characteristic property of the molecule; and (iv) calculating the biological characteristic property of the molecule by summing the contributions from the contributing substituent parts of the molecule. In this method, the contribution from a substituent part is equal to a function of the distance of the substituent part to the reaction center multiplied by a weight factor for the substituent part, and the same or substantially the same functional form for the function of the distance is used to calculate the contribution from each of the contributing substituent parts.

Another of the methods described in this patent is a method for calculating a biological characteristic property of a molecule by calculating the contributions from contributing substituent parts as described in the method above plus a contribution equal to a measured property of the molecule multiplied by a weight factor. Generally, the measured property of the molecule can be any property of the molecule that can be measured. In one version, the measured property may be the hydrophobicity of the molecule. In one version, the value of the hydrophobicity may be equal to the log of the octanol/water partition coefficient. In one version, the weight factor used in the calculation of the contribution from the measured property is calculated as a regression coefficient for a multivariate regression analysis calculated for a series of molecules.

In one version of the methods described in this patent, the methods may be used to calculate biological characteristic properties including but not limited to therapeutic index, effective dosage, inhibiting concentration, lethal dosage, hydrophobicity, solubility, toxicity, brain blood barrier crossing concentration, kinetics of biotransformation pathways, rate constant for in vivo or in vitro oxidation, rate constant for in vivo or in vitro phosphorylation, rate constant for in vivo or in vitro alkylation, rate constant for in vivo or in vitro glycosylation, absorption, clearance, metabolic stability, pharmacokinetics, t_{½} biological reactivity, bioefficacy, and binding affinity. Examples of effective dosages that may be calculated using the methods described in this patent include but are not limited to ED₅₀, ED₃₀, and ED₈₀. Examples of inhibiting dosages that may be calculated using the methods described in this patent include but are not limited to IC₅₀. Examples of lethal dosages that may be calculated using the methods described in this patent include but are not limited to LD₅₀ and LD₁₀₀.

In another version of the methods described in this patent, the methods may be used to calculate for a molecule a biological characteristic property that is characteristic of the interaction of the molecule with a subject organism or that is characteristic of the effect of the molecule on a subject organism. Subject organisms may be, but are not limited to, animal or a plant. Animal subject organisms may be, but are not limited to, mammals, which may be, but are not limited to human, mouse, guinea pig, rabbit, frog, dog and rat. Plant subject organisms may be, but are not limited to, soybean, corn, rice, wheat, canola, and potato. Other subject organism may be, but are not limited to, microorganisms, which may be, but are not limited, to bacteria, algae, archae and yeast. Other subject organisms may be, but are not limited to, fungi or viruses.

In another version of the methods described in this patent, the methods may be used to calculate for a molecule a biological characteristic property that is characteristic of the interaction of the molecule with or the effect of the molecule on cells, tissues, organs, organelles, or other portions of a subject organism. In this version, subject organisms may be, but are not limited to, the subject organisms described above.

In one version of the methods described in this patent, the methods may be used to calculate the biological characteristic property of organic molecules, inorganic molecules, neutral molecules, radicals, anions, cations, ionic salts, metallo-organic compounds, or coordination compounds. In specific versions, the methods may be used to calculate the biological characteristic property of aniline mustards, NSAIDs, or mitomycins.

Regarding the substituent parts of the molecule, in one version of the methods described in this patent, the substituent parts of the molecule may be atoms contained in the molecule or groups of connected atoms contained in the molecule.

Regarding the reaction center, generally the reaction center may be any point in space. In one version of the methods described in this patent, the reaction center may be a substituent part of the molecule which may be an atom contained in the molecule or may be a group of connected atoms contained in the molecule.

Regarding the contributing substituent parts of the molecule, generally any number of the substituent parts may make up the contributing substituent parts. In one version of the methods described in this patent, the contributing substituent parts include all substituent parts of the molecule except one. In another version of the methods described in this patent, the contributing substituent parts include all substituent parts in the molecule except the substituent part that is the reaction center.

Regarding the function of the distance used in the calculation of the contribution from a substituent part, generally this function may be of any functional form provided that the same or substantially the same functional form is used for calculating the contribution for each substituent part. In one version of the methods described in this patent, the function of the distance is an inverse function of the distance. In another version, the function of the distance goes as the inverse of the square of the distance. In another version, the function of the distance goes as the inverse of the cube of the distance. In another version, the function of the distance goes as the sum of the inverse of the square of the distance and the inverse of the cube of the distance.

Regarding the weight factor used in the calculation of the contribution from a substituent part, generally the weight factor may be calculated as a regression coefficient for a multivariate regression analysis calculated for a series of molecules. In one version of the methods described in this patent, the dependent variables for the multivariate regression analysis are the values of the biological characteristic property for the series of molecules and the independent variables are the distant dependent contribution for each type of substituent part present in the series of molecules. For a particular molecule in the series of molecules, the value of the independent variable corresponding to a particular type of substituent part is equal to a sum of the function of the distance from the reaction center to the particular substituent part, where the sum is over all occurrences of that particular substituent part. In one version of the methods described in this patent, the series of molecules include molecules that are analogs of the molecule for which the biological characteristic property is being calculated. In another version of the methods described in this patent, the series of molecules include molecules which include an atom or group of atoms that is the same as the reaction center of the molecule for which the biological characteristic property is being calculated.

Regarding how the reaction center may be selected, in one version of the methods described in this patent, the reaction center is selected by performing a multivariable regression analysis for two or more different possible reaction centers, calculating a characteristic of the multivariable regression analysis for each reaction center, and determining which reaction center corresponds to the multivariable regression analysis characteristic that satisfies a predetermined criteria. In one version of the methods described in this patent, the multivariable regression analysis characteristic is the global regression coefficient of the regression analysis and the predetermined criteria selects the reaction center with the highest global regression coefficient. In another version of the methods described in this patent, the multivariable regression analysis characteristic is the global standard error of the regression analysis and the predetermined criteria selects the reaction center with the lowest global standard error.

In addition to the methods describe above, other methods, devices, and compositions described in this patent include a computing device configured to calculate biological characteristic properties of molecules by one of the methods described in this patent; a computer-readable article of manufacture containing a computer program capable of being implemented in a computer to carry out one or more of the methods described in this patent; a molecule for which the structure was identified to include one or more substituent parts chosen to affect a biological characteristic property of the molecule, where the effect of the one or more substituent parts is calculated by one or more of the methods described in this patent; and a molecule synthesized after determining a likely biological characteristic property of the molecule, where the effect of the biological characteristic property of the molecule is calculated by one or more of the methods described in this patent.

### BRIEF DESCRIPTION OF THE SEVERAL VIEWS OF THE DRAWING(S)

FIG. 1. Predicted vs. Experimental ED₅₀ against Walker 256 Carcinoma in rats for aniline mustards.

FIG. 2. Predicted vs. Experimental LD₅₀ against Walker 256 Carcinoma in rats for aniline mustards.

FIG. 3. Predicted vs. Experimental Activity of Mitomycins, Expressed as log (1/C) Against Human Tumor Cells in Culture.

FIG. 4. Predicted vs. Experimental IC₅₀ (mmol/L) of NSAIDs against COX1.

FIG. 5. Predicted vs. Experimental IC₅₀ (mmol/L) of NSAIDs against COX2.

### DETAILED DESCRIPTION

The inventors have discovered new methods for calculating a biological characteristic property of a molecule by correlation analysis, and in this section we describe (1) specific aspects of the methods, (2) implementation of the methods in a computer system, (3) general uses of the methods, and (4) examples of results calculated using the methods.

Correlation Analysis Methods

The methods described in this patent may be used to calculate a biological characteristic property of a molecule. The biological characteristic properties that may be calculated and the classes of molecule to which the method may be applied are described in detail below. In the method, a molecule is conceptually separated into substituent parts, a reaction center is identified, and the distance of the substituent parts from the reaction center is calculated. The contribution from each substituent part is then calculated as a weight factor multiplied by a function of the distance of the substituent part from the reaction center. We describe in detail below the various forms of distant dependent function that may be used and the various methods that may be used for identifying the reaction center and calculating the weight factor.

In terms of an equation, the method may be written as

$$BCP = {\sum }_{j = 1}^{n} {W}_{j} f ( {r}_{j} ) \qquad ( 3 )$$

where BCP is the value of the biological characteristic property of the molecule, the sum over j is a sum over the substituent parts of the molecule, Wⱼ is the weight factor associated with substituent j, rⱼ is the distance from substituent j to the reaction center and f(rⱼ) is a function of the distance from substituent j to the reaction center.

In one version of the methods described in this patent, BCP is the value of the biological characteristic property measured relative to some constant value, which in this patent we denote by BCP⁰. In one version, BCP⁰ may be the value of the biological characteristic property for a standard compound. In another version, BCP⁰ may be the value of the intercept of a multiple regression analysis, as will be described in detail elsewhere in this patent.

In another version of the methods described in this patent, a biological characteristic property of a molecule is equal to the contributions of the substituent parts as described above plus a contribution from one or more measured properties of the molecule. The contribution from a measured property is equal to the value of the measured property multiplied by a weight factor. We describe in detail below measured properties of the molecule that may be used and methods that may be used for calculating the weight factor.

In terms of an equation, this method may be written as

$$BCP = {\sum }_{j = 1}^{n} {W}_{j} f ( {r}_{j} ) + {\sum }_{k = 1}^{m} {w}_{k} M {P}_{k} \qquad ( 4 )$$

where the sum over k is a sum over the measured properties of the molecule, wₖ is the weight factor associated with the measured property k, and MPₖ is the value of measured property k.

Molecules for Which Biological Characteristic Properties May be Calculated

Generally, the methods described in this patent may be used to calculate the biological characteristic properties of any molecules and molecular fragments, including but not limited to organic molecules, inorganic molecules, neutral molecules, radicals, anions, cations, ionic salts and metallo-organic and coordination compounds. In one version of the methods described in this patent, the methods may be used to calculate the biological characteristic properties of peptides, proteins, and non-peptide small molecules. The methods described in this patent may be used to calculate the biological characteristic properties of molecules of arbitrary size. In another version of the methods described in this patent, the methods may be used to calculate biological characteristic properties for aniline mustards, nonsteroidal anti-inflammatory drugs (NSAID), and mitomycins. In another version of the methods described in this patent, the methods may be used to calculate biological characteristic properties for amines, or carboxylic acids.

As will be described in detail below, the methods described in this patent include a function of the distances of substituent parts from a reaction center. To facilitate this calculation, the 3D structure of the molecule may be obtained by any method capable of providing the 3D structure, including but not limited to theoretical modeling calculations, experimental x-ray diffraction data, and other experimental data, such as NMR data. In one version of the methods described in this patent, the 3D structure is obtained by using the Hyperchem software package available from HyperCube, Inc.

Biological Characteristic Properties that may be Calculated

Generally, any biological characteristic properties that can be measured may be calculated by the methods described in this patent. As used in this patent, “biological characteristic property” of a molecule means generally any property of a molecule that may have an affect on a biological system or is any property of a biological system affected by a molecule. The biological property may be measured at the molecular level (for example, hydrophobicity or rate constants for oxidation), at the cellular level (for example, in vitro cellular parameters) or at the organism system level (for example, therapeutic index). Examples of biological characteristic properties that may be calculated by the methods described in this patent include, but are not limited to, therapeutic index, effective dosage (ED), inhibiting concentration (IC), lethal dosage (LC), hydrophobicity, solubility, toxicity, brain blood barrier crossing concentration, kinetics of biotransformation pathways, rate constant for in vivo or in vitro oxidation, rate constant for in vivo or in vitro phosphorylation, rate constant for in vivo or in vitro alkylation, and rate constant for in vivo or in vitro glycosylation, absorption, clearance/metabolism, metabolic stability, pharmacokinetics, and t_{½} biological reactivity. Further examples of biological properties include bioefficacy, binding affinity, ED₅₀, ED₃₀, or ED₈₀, IC₅₀, or LD₁₀₀, or LD₅₀.

In another version of the methods described in this patent, the methods may be used to calculate biological characteristic properties that are characteristic of the interaction of the molecule with a subject organism such as an animal or plant. In one version, the biological characteristic property may be characteristic of the interaction of the molecule with mammals including, but not limited to, humans, dogs, mice, guinea pigs, rabbits, frogs, or rats. In another version, the biological property calculated can be characteristic of the interaction of the molecule with soybean, corn, rice, wheat, canola, or potato plants. The method can also be used to calculate properties of a molecule including those characteristic of the interaction of the molecule with tissues, cells, organs, organelles, or other portions of a biological system. In another version, the biological characteristic property may be characteristic of the interaction of the molecule with yeast, fungi, bacteria, plants, algae, viruses, archae, or bacteria.

Methods of Calculation of Biological Characteristic Property

In one version of the methods described in this patent, the biological characteristic property is calculated as the sum of contributions from substituent parts of the molecule. As described below in detail, not all substituent parts of the molecule need be included in this calculation. In this version, the biological characteristic property is calculated as equal to a sum of contributions from each contributing substituent part and the contribution of each substituent part is equal to the product of a weight factor multiplied by a function of the distance of the substituent part to a reaction center.

This version of the methods described in this patent is shown in equation form in equation 3 above.

In another version of the methods described in this patent, a biological characteristic property of a molecule is equal to the contributions of the substituent parts as described above plus a contribution from one or more measured properties of the molecule. The contribution from a measured property is equal to the value of the measured property multiplied by a weight factor. We describe in detail below measured properties of the molecule that may be used and methods that may be used for calculating the weight factor.

This version of the methods described in the patent is shown in equation form in Equation 4 above.

Substituent Parts

As part of the methods described in this patent, a molecule is conceptually separated into substituent parts and the biological characteristic property is calculated as the sum of contribution from some number of the substituent parts. The substituent parts contributing to the calculation of the biological characteristic property are referred to in this patent as the “contributing substituent parts.” Generally, the substituent parts of a molecule may be any portion of the molecule, including but not limited to, individual atoms in the molecule, groups of atoms in the molecule, individual portions of high electron density in the molecule (for example, lone pairs). In one version of the methods described in this patent, the substituent parts are individual atoms or groups of atoms. A person well versed with the use of correlation analysis to calculate the properties of molecules will understand how to identify atoms and groups that may be used as substituent parts. Generally, however, any portion of the molecule, including atoms and groups may be used as substituent parts.

Non-limiting examples of atoms and groups that may be used as substituent parts include all possible atoms, alkyl groups, alkenyl groups, aromatic groups, metallo-organic groups, and hetero-aromatic groups. A person familiar with the technology of correlation analysis will be able in a straight forward manner to identify other groups that may be used.

Generally, any number of the substituent parts may be contributing substituent parts. In one version, all of the substituent parts except one are contributing substituent parts. In another version in which the reaction center is a substituent part, all of the substituent parts except the reaction center are contributing substituent parts. In a version in which the contribution of a substituent part diminishes as the distance to the reaction center increases, substituent parts distant from the reaction center may make insignificant contribution to the calculated property and may be omitted from the contributing substituent parts. Such distant substituent parts may, however, also be included in the contributing substituent parts.

Reaction Center

In the methods described in this patent, having determined the contributing substituent parts of the molecule, one then calculates the distance from the contributing substituent parts to a reaction center. Generally, the reaction center can be any point in space. As will be described below in detail, in one version of the methods described in this patent an optimal reaction center may be identified by varying the position of the reaction center, calculating the weight factors for the substituent parts by multivariable regression analysis using the various reaction centers, and identifying the optimal reaction center as that center yielding the best regression analysis fit. In one version, the reaction center may be identified as one of the substituent parts of the molecule.

Functional Forms

The inventors have discovered that it is possible to take into account the structure of a molecule when calculating a biological characteristic property if the contribution of each contributing substituent part is proportional to a function of the distance of the substituent part to the reaction center. The function of the distance used to calculate the contribution for each substituent has the same or substantially the same functional form; the function of the distance may, however, generally be of any functional form. By substantially the same functional form, we mean a functional form that is not identical to the other functional forms but for which the difference in functional form does not qualitatively affect the results of the calculations. As a nonlimiting example, functional forms of 1/r² and 1/r^{(2+δ)} may be considered substantially the same for small δ.

In one version of the methods described in this patent, the functional form is a function of the inverse of the distance. In another version, the functional form goes as the inverse of the square of the distance (i.e., f(r) proportional to 1/r²). In another version, the functional form goes as the inverse of the cube of the distance (i.e., f(r) proportional to 1/r³). In another version, the functional form goes as 1/r²+1/r³.

In the 1/r² version, for example, equation (3) becomes:

$$BCP = {\sum }_{j = 1}^{n} \frac{{W}_{j}}{{r}_{j}^{2}}$$

Calculation of the Weight Factors

As part of the methods described in this patent, the contribution to the characteristic property of a molecule by a substituent part is given by a function of the distance of that substituent part from a reaction center multiplied by a weight factor. Generally the weight factor may be calculated as a regression coefficient for a multivariate regression analysis calculated for a series of molecules. Below we describe one specific version of the methods that may be used to calculate the weight factors, but first we describe in more general terms methods that may be used. A description of the implementation of multivariate regression analysis may be found in for example Essentials of Statistics, Stephen A. Book, New York, McGraw Hill, 1978, page 315 et seq.

In one version of the methods described in this patent, the dependent variables for the multivariate regression analysis are the values of the characteristic property for the series of molecules and the independent variables are the distant dependent contribution for each type of substituent part present in the series of molecules. For a particular molecule in the series of molecules, the value of the independent variable corresponding to a particular type of substituent part is equal to a sum of the function of the distance from the reaction center to the particular substituent part, where the sum is over all occurrences of that particular substituent part. In one version of the methods described in this patent, the series of molecules include molecules that are analogs of the molecule for which the characteristic property is being calculated. In another version of the methods described in this patent, the series of molecules include molecules which include an atom or group of atoms that is the same as the reaction center of the molecule for which the characteristic property is being calculated.

One specific example of the multivariable regression analysis that may be used to calculate the weight factors is as follows. This example calculates the weight factors for a version of the methods described in this patent in which the function of the distance used in calculating the contribution of the substituent parts goes as one over the inverse of the distance. In a more general version of the methods described in this patent in which the function of the distance may be any function, f(r), the following example will still apply except that the R-matrix contains terms of the form

$${\sum }_{k} f ( {r}_{rc - mk} )$$

rather than

$${\sum }_{k} \frac{1}{{r}_{rc - {m}_{k}}^{2}} .$$

This example is presented in three steps: first, calculation of the geometries of the series of molecules used to calculate the weights; second, the calculations of the “R-matrix;” and third, the multivariable regression analysis, also called the partial least squares analysis, used to calculate the weights as the regression coefficients.

1. Input. Structural files for optimized geometries of molecules of reaction series are prepared, where each contributing substituent part is specified with its number and 3 spatial coordinates.

If a reaction series contains M molecules, then the input of M structural files should be prepared. For each molecule j, its, reaction center (rcⱼ) is specified by placing the corresponding atomic number into [rc_{i.}, . . . , _{.}rc_{j.}, . . . , rc_{M}]−vector.

2. R-Matrix. The next step of the procedure is composition of the R-matrix containing sums of the

$${\sum }_{k} \frac{1}{{r}_{rc - {m}_{k}}^{2}}$$

terms, related to certain types of substituent parts.

When there are K types of substituent parts presented in molecules of the reaction series, the [M×K] R-matrix is formed. For each structural file the program sorts the atoms according to specified types of substituent parts and calculates the sums

$${\sum }_{k} \frac{1}{{r}_{rc - {m}_{k}}^{2}} ,$$

where r is the direct distance between substituent parts of m-type in molecule j and the reaction center and k sums over the substituent parts of type m in the molecule j:

$$R = [ \begin{matrix}{( {\sum }_{k} \frac{1}{{r}_{rc - {m}_{k}}^{2}} )}_{1 , 1} {( {\sum }_{k} \frac{1}{{r}_{rc - {m}_{k}}^{2}} )}_{1 , 2} {⋯ ( {\sum }_{k} \frac{1}{{r}_{rc - {m}_{k}}^{2}} )}_{1 , K} \\ {( {\sum }_{k} \frac{1}{{r}_{rc - {m}_{k}}^{2}} )}_{j , 1} {( {\sum }_{k} \frac{1}{{r}_{rc - {m}_{k}}^{2}} )}_{j , 2} {⋯ ( {\sum }_{k} \frac{1}{{r}_{rc - {m}_{k}}^{2}} )}_{j , K} \\ {( {\sum }_{k} \frac{1}{{r}_{rc - {m}_{k}}^{2}} )}_{M , 1} {( {\sum }_{k} \frac{1}{{r}_{rc - {m}_{k}}^{2}} )}_{M , 2} {⋯ ( {\sum }_{k} \frac{1}{{r}_{rc - {m}_{k}}^{2}} )}_{M , K}\end{matrix} ]$$

In the absence of contributing substituent parts of m-type in the molecule n, the corresponding matrix element is set equal to 0:

3. Partial Least Square (PLS)—analysis. The final step in this procedure is estimation whether the dataset can be treated as set dependent parameters of multilparameter regression with an intercept equal to BCP⁰ For example, when the method of the invention is applied to free energy (ΔG is the free energy measured relative to some standard free energy G⁰), the experimental parameters of free energy changes are taken as the vector ΔG:

$$Δ G = [ \begin{matrix}Δ {G}_{1} \\ Δ {G}_{2} \\ … \\ Δ {G}_{M}\end{matrix} ] ,$$

the equation can be written in matrix notation as the following:

R g=ΔG

where g is solution vector

$$[ \begin{matrix}{g}_{1} \\ {g}_{2} \\ … \\ {g}_{K}\end{matrix} ] ,$$

containing K values of what will be the weight factors (Wⱼ) which here are designated gᵢ, corresponding to all types of contributing substituent parts.

When M>K (i.e. the number of molecules in reaction series is greater then the number of types of contributing substituent parts) the system is consistent and R g=ΔG can be solved.

An approximate solution of equation can be achieved by multivariable regression, when the columns of R—matrix are considered as sets of independent variables and set ΔG values as dependent parameters. If such regression can be estimated with high accuracy, its linear coefficients can be taken as the weight factors, corresponding to the types of contributing substituent parts.

Additional Measured Properties That May Contribute to the Calculated Biological Characteristic Property and Calculation of Weights for the Additional Measured Properties

As presented in Equation 4 above and the supporting description, in one aspect of the methods described in this patent, the biological characteristic property is calculated as a contribution from the contributing substituent parts plus a contribution from one or more measured properties of the molecule. In one version of these methods, there is a contribution from one measured property of the molecule. Generally, any property of the molecule may be included as a measured property. Properties that may be measured properties include but are not limited to biological properties, chemical properties, and physical properties of the molecule. In one version, the hydrophobicity of the molecule is one measured property that may be used. In one version, the hydrophobicity may be calculated as the logarithm of the octanol-8/water partition coefficient.

Implementation of the Methods

The methods described in this patent may be implemented using any device capable of implementing the methods. Examples of devices that may be used include but are not limited to electronic computational devices, including computers of all types. When the methods described in this patent are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, world wide web, an intranet, or other network.

In one example, the methods described in this patent may be implemented in a system comprising a processor and a computer readable medium that includes program code means for causing the system to carry out the steps of the methods described in this patent. The processor may be any processor capable of carrying out the operations needed for implementation of the methods. The program code means may be any code that when implemented in the system can cause the system to carry out the steps of the methods described in this patent. Examples of program code means include but are not limited to instructions to carry out the methods described in this patent written in a high level computer language such as C++, Java, or Fortran; instructions to carry out the methods described in this patent written in a low level computer language such as assembly language; or instructions to carry out the methods described in this patent in a computer executable form such as compiled and linked machine language.

Uses of the Methods

The methods described in this patent may be used in a variety of ways including but not limited to the prediction of a biological characteristic property of a molecule that has not previously been synthesized or for which the biological characteristic property has not previously been measured; investigation of the effect of structural modification on the biological characteristic property of a molecule, which may be used to identify candidate molecules for use in specific circumstances, including but not limited to uses as pharmaceuticals. The methods described in this patent may be used to predict the biological characteristic properties of any molecule or molecule fragment for which the structure is known or may be obtained. The methods may be used to predict the efficacy of a molecule or molecular fragment for various uses including but not limited to use as a pharmaceutical, herbicide, insecticide, nutraceutical, cosmetic, or fungicide.

### EXAMPLE

The following examples demonstrate implementation of various methods described in this patent and demonstrate the operability and utility of these methods. The general approach in these examples is to compose a matrix [M×K] r⁻² of a series of molecules (M) containing a number of different types of contributing substituent parts (K). The interatomic distances, r, are determined by using the Hyperchem software package, which allows simple estimation of standard geometries of the corresponding molecules. The resulting r⁻² matrices are then analyzed with the appropriate multivariable regression analysis to determine the weight parameters. The implementation of this method is referred to in these examples as the 3D-CAN(TM) method. In these examples the contributing substituent parts are referred to as “atomic types” or some similar phrase, and the weight factors are referred to as “operational parameters,” “operational atomic parameters,” or similar phrase and are designated edᵢ, 1dᵢ, gₗ, icᵢ, cox1ᵢ; and cox2ₗ in the various examples. Methods described in these examples that include a contribution from a measured property of the molecule are referred to as “modified 3D-CAN(TM)” or similar phrase.

The examples below demonstrate the calculation of biological characteristic properties using both a method that does not include a contribution from a measured property of the molecule (example 1 and 3) and a method that does include a contribution from a measured property (hydrophobicity) of the molecule (examples 2 and 3). The examples below also demonstrate specific implementation of methods that may be used in the selection of a reaction center (examples 2 and 3).

As used in these examples, an atom designation of C4 for example represents a 4-coordinate carbon atom (i.e., sp³ hybridized), C3 represents a 3-coordinate carbon atom (i.e., sp² hybridized), N3 represents a 3-coordinate nitrogen atom (i.e., sp² hybridized), etc.

### Example 1

### Application of the Modified 3D CAN(TM) to Quantification of Therapeutic Index for a Series of Aniline Mustards

In order to illustrate the possibilities of 3D-CAN(TM) as an effective tool of molecular modeling and drug design, we have considered the series of DNA-linking reagents—aniline mustards 4-R—C₆H₄—N(C₂H₄Cl)₂, acting as an effective anticancer drugs (Gupta, Chemical Reviews (1994)). The central mustard nitrogen (marked with an *) was defined as the reactive center for the purpose of these calculations.

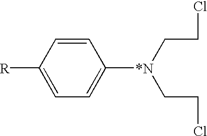

Their activity (ED₅₀) against Walker 256 Carcinoma in rats and their toxicity (LD₅₀), presented in Table 1 below, have been evaluated in the framework of 3D-CAN(TM).

TABLE 1
Experimental and Predicted Activity and Toxicity for Aniline Mustards
Modeled with 3D CAN(TM)

| | | log(1/ED₅₀) | log(1/ED₅₀) | log(1/LD₅₀) | log(1/LD₅₀) |
|---|---|---|---|---|---|
| Nr | R | experiment | prediction | experiment | prediction |
| 1 | H | 3.4 | 3.48 | 3.44 | 3.61 |
| 2 | COO⁻ | 3.3 | 3.29 | 3.04 | 3.04 |
| 3 | SO₂NH₂ | 2.82 | 2.82 | 2.95 | 2.95 |
| 4 | OH | 4.49 | 4.46 | 4.13 | 4.24 |
| 5 | NH₂ | 4.7 | 4.53 | 4.82 | 4.76 |
| 6 | NHCOCH₃ | 4.47 | 4.56 | 3.99 | 3.76 |
| 7 | NHCOCH₂NH₂ | 4.47 | 4.81 | 4.47 | 4.29 |
| 8 | NHCOCH₂NH—COCH₃ | 4.8 | 4.29 | 4.17 | 4.39 |
| 9 | NHCOCH₂NH—COOCH₃ | 3.85 | 4.05 | 3.7 | 3.82 |
| 10 | OCOCH₃ | 4.58 | 4.56 | 4.26 | 4.05 |
| 11 | OCOCH₆H₅ | 4.82 | 4.89 | 4.03 | 4.10 |
| 12 | OCOC₆H₃-2,6-(CH₃)₂ | 3.27 | 3.36 | 3.07 | 3.11 |
| 13 | OCOC₆H₄-2-(CH₃) | 4.51 | 4.33 | 3.68 | 3.61 |
| 14 | 4-C₆H₄—OCONH—C₆H₄-4-COO⁻ | | | 2.93 | 2.89 |

Parameters of the effective dosage log(1/ED₅₀) and toxicity log(1/LD₅₀) for compounds 1-14 have been analyzed within 3D-CAN(TM)—equations:

$$log ( \frac{1}{{ED}_{50}} ) = {a}_{0} + {\sum }_{i \neq rc}^{N - 1} \frac{{ed}_{i}}{{r}_{rc - i}^{2}}$$

$${a}_{0} = - 73.4 , N = 13 , S = 0.66 , r = 0.9601 ;$$

$$log ( \frac{1}{{LD}_{50}} ) = {a}_{1} + {\sum }_{i \neq rc}^{N - 1} \frac{{ld}_{i}}{{r}_{rc - i}^{2}}$$

$${a}_{1} = 8.8 , N = 14 , S = 0.53 , r = 0.9733 ;$$

where N is number of atoms in molecule, r is the distance between i-th atom and the reaction center (nitrogen) and a₀, a₁, are standard values, ed and ld are introduced 3D-CAN(TM) operational atomic parameters, depending on the nature of atom and its valent state.

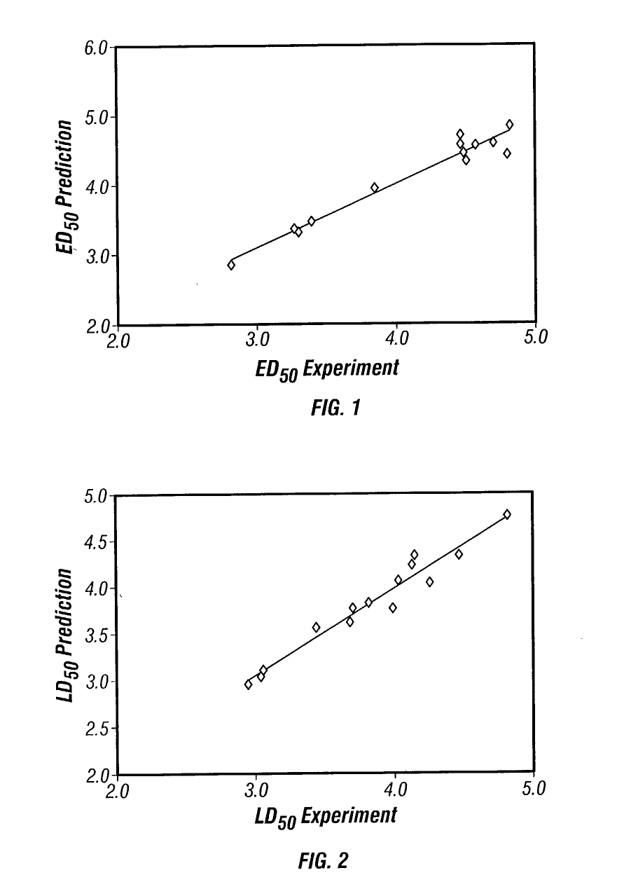

Correlations for the above equations have been estimated with high accuracy and presented in graphic form on FIGS. 1 and 2 respectively. The predicted values of log(1/ED₅₀) and log(1/LD₅₀) are given and Table 1. Operational parameters estimated for atomic types and are presented in Table 2.

TABLE 2
Operational atomic parameters ed and ld

| Atomic type | ed | ld |
|---|---|---|
| H | −369.2 | −69.9056 |
| C4 | 644.4 | 75.3455 |
| C3 | −495.6 | −492.887 |
| Cₐᵣ | 214.2 | 28.4115 |
| O2 | 25.7 | 43.3476 |
| O═ | 376.5 | 445.9569 |
| Cl | 561.1 | 210.4722 |
| S6 | −789.1 | −934.686 |
| O⁻ | −1299.1 | −136.24 |
| N2 | 383.8 | 143.6177 |

These data show that the methods described in this patent may be used to predict unknown values of ED₅₀ and LD₅₀ for mustards, composed from atomic types, given in Table B. For the investigated anticancer drugs, their anti-tumor activity 1/ED₅₀ is expected to be as high as possible. In the same time, their toxicity 1/LD₅₀ should be suppressed. The therapeutic index (LD₅₀/ED₅₀) for 4-substituted aniline mustards under study are given in the Table 3 below.

TABLE 3
Selectivity ratio LD₅₀/ED₅₀ for 4-substituted
aniline mustards.

| Nr | [0001] R | LD₅₀/ED₅₀ |
|---|---|---|
| 1 | H | 0.912011 |
| 2 | COOH | 1.819701 |
| 3 | SO₂NH₂ | 0.74131 |
| 4 | OH | 2.290868 |
| 5 | NH₂ | 0.758578 |
| 6 | NHCOCH₃ | 3.019952 |
| 7 | NHCOCH₂NH₂ | 1 |
| 8 | NHCOCH₂NH—COCH₃ | 4.265795 |
| 9 | NHCOCH₂NH—COOCH₃ | 1.412538 |
| 10 | OCOCH₃ | 2.089296 |
| 11 | OCOC₆H₅ | 6.16595 |
| 12 | OCOC₆H₃-2,6-(CH₃)₂ | 1.584893 |
| 13 | OCOC₆H₄-2-(CH₃) | 6.76083 |
| 14 | OCONH—C₆H₄-4-COOH | 16.98244 |

Based on the estimated parameters ed and Id, we can demonstrate that the substitution of aniline mustard C₆H₅—N(C₂H₄Cl)₂ in para-position by OCONH—C₆H₄-4-COO⁻-group will likely yield significantly increased 1/ED₅₀ for this compound, while the corresponding 1/LD₅₀ value should not rise dramatically. The calculated values of 1/ED₅₀ and 1/LD₅₀ for the modeled compound are 5.06 and 3.83 respectively. The corresponding experimental values have bee estimated as 5.05 and 3.82. Therefore, the designed compound, being the most active, is also the most selective. It is 17-fold more effective against tumor cells relatively to normal, while for other similar drugs the best selectivity ratio could be achieved as low as 6-7. This demonstrates that 3D-CAN(TM) may effectively be used for actual design of compounds with desired properties.

### Example 2

### Application of the Modified 3D CAN(TM) to Quantification of Mitomycin Series of Anti-Cancer Compounds

In order to evaluate the applicability of the developed approach for quantification of bioactivity data we have considered anti tumor activity of substituted mytomycins. A number of attempts have been previously made to study structure-activity relationships of mytomycins—clinical antitumor agents of the quinone series.

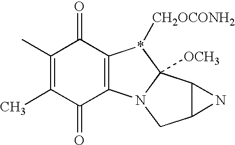

No satisfying results have previously been obtained. The best correlation could be estimated between activity of compounds 1-30 (See Table 7) and the corresponding values of their logP and redox potentials. The coefficient of the correlation has been established as 0.84.

We have considered a number of derivatives of Mitomycin C (1-19) and Mitomycin A (20-30) and processed their activities (expressed in concentration C which is average IC₅₀ from assays) against human tumor cells in culture (S. P. Gupta, Chem. Review, 94, No. 6, 1519 (1994)). The corresponding experimental log(1/C) and logP values have been processed within the modified 3D CAN(TM) schemata, where the parameters are modeled as the following:

$$log ( \frac{1}{C} ) = const + {\sum }_{i \neq rc}^{N - 1} \frac{{g}_{i}}{{r}_{rc - i}^{2}} + α log P$$

where N is the number of atoms in the molecule,

r_{rc-i} is the distance between atom i and the reaction center (rc) and icₗ is introduced operational atomic parameters, reflecting the ability of an atom of a certain type to contribute into overall 1/C−value.

logP is the empirical measure of hydrophobicity.

Since the equation above contains intraatomic distance to the atom selected as a reaction center, 3D CAN(TM) allows scanning multiple potential reaction centers to establish the appropriate one, based on the quality of the regression. Several common atoms were tested as a potential reaction center of the series.

For the mytomycins series we have considered numerous common atoms as a potential reaction centers (rc). For example, when the carbon atom of the quinolone o-methyl group has been considered as the reaction center, the quality of the regression is poor as can be seen in the following table:

Regression Statistics

| | |
|---|---|
| Multiple R | 0.890038 |
| R Square | 0.792167 |
| Adjusted R Square | 0.536372 |
| Standard Error | 0.542012 |
| Observations | 30 |

The corresponding atomic operational parameters also have poor quality (see Table 4.)

TABLE 4
Operational Parameters for Atomic Group Using the
Quinolone Carbon as RC

| Atomic type | Coefficients | Standard Error |
|---|---|---|
| Const | −7.09212 | 14.77619 |
| H | 22.58408 | 11.8968 |
| C4 | −31.9739 | 16.13538 |
| C═ | −25.7366 | 14.09525 |
| C aromatic | −9.3124 | 6.767811 |
| N3 | −102.108 | 14.5592 |
| —O— | −64.1973 | 9.511758 |
| O═ | 377.0665 | 119.1042 |
| F | 5.937482 | 30.53861 |
| Br | 11.06703 | 34.05972 |
| I | 17.64792 | 27.12964 |
| —S— | −16.3543 | 9.743814 |
| —N═ | 173.6192 | 61.12753 |
| N nitro | −645.49 | 205.5137 |
| N indole | 18.09392 | 33.21112 |
| N pyridine | −27.5241 | 27.39797 |

The best quality regression parameters were obtained when an atom in the center ring of mytomycin (marked with a star in the structure above) was considered as the rc. The parameters of the corresponding regression, estimated in this approximation are presented in following table:

Regression Statistics

| | |
|---|---|
| Multiple R | 0.956692 |
| R Square | 0.91526 |
| Adjusted R Square | 0.810965 |
| Standard Error | 0.346095 |
| Observations | 30 |

When the hydrophobicity is not taken into account, the quality of the correlation is lower:

Regression Statistics

| | |
|---|---|
| Multiple R | 0.949617 |
| Adjusted R Square | 0.796527 |
| Standard Error | 0.359069 |
| Observations | 30 |

The estimated atomic operational contributions determined by regression are given in Table 5 and the operational R matrix of the modified 3D CAN(TM) (matrix of parameters) is given as Table 6.

TABLE 5
Operational atomic parameters g, derived for the
presented atomic types.

| | Coefficients | Standard Error |
|---|---|---|
| const | −3.22439 | 14.49385 |
| H | 27.2439 | 11.9157 |
| C4 | −41.5106 | 16.90643 |
| C═ | −39.3292 | 16.5488 |
| C aromatic | −15.2638 | 7.724581 |
| N3 | −95.8146 | 14.6992 |
| —O— | −54.2981 | 11.46341 |
| O═ | 420.8054 | 118.7589 |
| F | 8.571243 | 29.49205 |
| Br | 2.105548 | 33.4149 |
| I | 3.576405 | 27.91911 |
| —S— | −18.4213 | 9.501031 |
| —N═ | 207.4299 | 63.43391 |
| N nitro | −714.328 | 203.7863 |
| N indole | 17.87198 | 32.01148 |
| N pyridine | −28.297 | 26.41347 |
| logP | 0.211075 | 0.146731 |

Table 6
The operational R matrix of the modified 3D CAN(TM)
(matrix of parameters) ]

| Compound/ | | | | C aro- | | | | |
|---|---|---|---|---|---|---|---|---|
| Atomic type | H | C4 | C═ | matic | N3 | —O— | O═ | F |
| 1 | 2.1452 | 1.3313 | 1.0476 | 0.0000 | 0.3369 | 0.1745 | 0.2157 | 0.0000 |
| 2 | 2.2659 | 1.4152 | 1.0556 | 0.0000 | 0.3282 | 0.1867 | 0.2148 | 0.0000 |
| 3 | 2.2092 | 1.3681 | 1.0852 | 0.0000 | 0.3376 | 0.1746 | 0.2196 | 0.0000 |
| 4 | 2.2637 | 1.4374 | 1.0477 | 0.0000 | 0.3374 | 0.1916 | 0.2195 | 0.0000 |
| 5 | 2.2376 | 1.3901 | 1.1043 | 0.0000 | 0.3370 | 0.1892 | 0.2213 | 0.0000 |
| 6 | 2.2929 | 1.3999 | 1.0482 | 0.0812 | 0.3375 | 0.1744 | 0.2157 | 0.0000 |
| 7 | 2.2096 | 1.3344 | 1.0479 | 0.1538 | 0.3369 | 0.1745 | 0.2194 | 0.0000 |
| 8 | 2.2197 | 1.3333 | 1.0481 | 0.1536 | 0.3508 | 0.1742 | 0.2195 | 0.0000 |
| 9 | 2.1954 | 1.3344 | 1.0483 | 0.1532 | 0.3369 | 0.1745 | 0.2195 | 0.0140 |
| 10 | 2.1952 | 1.3344 | 1.0484 | 0.1536 | 0.3369 | 0.1745 | 0.2195 | 0.0000 |
| 11 | 2.1965 | 1.3342 | 1.0481 | 0.1540 | 0.3368 | 0.1744 | 0.2192 | 0.0000 |
| 12 | 2.1953 | 1.3344 | 1.0483 | 0.1542 | 0.3367 | 0.1745 | 0.2194 | 0.0000 |
| 13 | 2.2078 | 1.3342 | 1.0480 | 0.1535 | 0.3368 | 0.1884 | 0.2195 | 0.0000 |
| 14 | 2.1945 | 1.3338 | 1.0483 | 0.1542 | 0.3365 | 0.1747 | 0.2441 | 0.0000 |
| 15 | 2.1949 | 1.3341 | 1.0483 | 0.1535 | 0.3366 | 0.1886 | 0.2196 | 0.0000 |
| 16 | 2.1932 | 1.3324 | 1.0478 | 0.1525 | 0.3365 | 0.1884 | 0.2411 | 0.0000 |
| 17 | 2.2053 | 1.3330 | 1.0481 | 0.1908 | 0.3365 | 0.1744 | 0.2193 | 0.0000 |
| 18 | 2.1513 | 1.3501 | 1.1238 | 0.0000 | 0.3370 | 0.1749 | 0.2186 | 0.0000 |
| 19 | 2.1722 | 1.3334 | 1.1414 | 0.0000 | 0.3558 | 0.1745 | 0.2152 | 0.0000 |
| 20 | 2.1814 | 1.3796 | 1.0562 | 0.0000 | 0.2871 | 0.2147 | 0.2170 | 0.0000 |
| 21 | 2.2248 | 1.4370 | 1.0559 | 0.0000 | 0.2869 | 0.2147 | 0.2183 | 0.0000 |
| 22 | 2.3145 | 1.4789 | 1.0561 | 0.0000 | 0.2868 | 0.2153 | 0.2169 | 0.0000 |
| 23 | 2.3140 | 1.4785 | 1.0563 | 0.0000 | 0.2868 | 0.2152 | 0.2175 | 0.0000 |
| 24 | 2.2195 | 1.3776 | 1.0558 | 0.0895 | 0.2868 | 0.2151 | 0.2164 | 0.0000 |
| 25 | 2.2381 | 1.4093 | 1.0558 | 0.0000 | 0.2869 | 0.2376 | 0.2170 | 0.0000 |
| 26 | 2.2359 | 1.3998 | 1.0558 | 0.0562 | 0.2869 | 0.2323 | 0.2171 | 0.0000 |
| 27 | 2.2476 | 1.4230 | 1.0563 | 0.0000 | 0.2870 | 0.2422 | 0.2171 | 0.0000 |
| 28 | 2.2615 | 1.4309 | 1.0556 | 0.0000 | 0.2871 | 0.2428 | 0.2165 | 0.0000 |
| 29 | 2.2319 | 1.3992 | 1.0561 | 0.0496 | 0.2870 | 0.2149 | 0.2168 | 0.0000 |
| 30 | 2.2327 | 1.4170 | 1.0559 | 0.0000 | 0.2869 | 0.2224 | 0.2169 | 0.0000 |
| Compound/ | | | | | | N | N | |
| Atomic type | Br | I | —S— | —N═ | N nitro | indole | pyridine | logP |
| 1 | 0.0000 | 0.0000 | 0.0000 | 0.0000 | 0.0000 | 0.0000 | 0.0000 | −0.38 |
| 2 | 0.0000 | 0.0000 | 0.0000 | 0.0000 | 0.0000 | 0.0000 | 0.0000 | 0.1 |
| 3 | 0.0000 | 0.0000 | 0.0000 | 0.0000 | 0.0000 | 0.0000 | 0.0000 | 0.24 |
| 4 | 0.0000 | 0.0000 | 0.0000 | 0.0000 | 0.0000 | 0.0000 | 0.0000 | 0.21 |
| 5 | 0.0000 | 0.0000 | 0.0000 | 0.0000 | 0.0000 | 0.0000 | 0.0000 | 1.9 |
| 6 | 0.0000 | 0.0000 | 0.0000 | 0.0000 | 0.0000 | 0.0000 | 0.0177 | 1.23 |
| 7 | 0.0000 | 0.0000 | 0.0000 | 0.0000 | 0.0000 | 0.0000 | 0.0000 | 1.3 |
| 8 | 0.0000 | 0.0000 | 0.0000 | 0.0000 | 0.0000 | 0.0000 | 0.0000 | 0.07 |
| 9 | 0.0000 | 0.0000 | 0.0000 | 0.0000 | 0.0000 | 0.0000 | 0.0000 | 1.44 |
| 10 | 0.0126 | 0.0000 | 0.0000 | 0.0000 | 0.0000 | 0.0000 | 0.0000 | 2.16 |
| 11 | 0.0000 | 0.0113 | 0.0000 | 0.0000 | 0.0000 | 0.0000 | 0.0000 | 2.42 |
| 12 | 0.0000 | 0.0122 | 0.0000 | 0.0000 | 0.0000 | 0.0000 | 0.0000 | 2.42 |
| 13 | 0.0000 | 0.0000 | 0.0000 | 0.0000 | 0.0000 | 0.0000 | 0.0000 | 0.63 |
| 14 | 0.0000 | 0.0000 | 0.0000 | 0.0000 | 0.0137 | 0.0000 | 0.0000 | 1.02 |
| 15 | 0.0000 | 0.0113 | 0.0000 | 0.0000 | 0.0000 | 0.0000 | 0.0000 | 1.75 |
| 16 | 0.0000 | 0.0000 | 0.0000 | 0.0000 | 0.0126 | 0.0000 | 0.0000 | 0.51 |
| 17 | 0.0000 | 0.0000 | 0.0000 | 0.0000 | 0.0000 | 0.0146 | 0.0000 | 2.45 |
| 18 | 0.0000 | 0.0000 | 0.0365 | 0.0177 | 0.0000 | 0.0000 | 0.0000 | 1.52 |
| 19 | 0.0000 | 0.0000 | 0.0000 | 0.0220 | 0.0000 | 0.0000 | 0.0000 | 0.56 |
| 20 | 0.0000 | 0.0000 | 0.0000 | 0.0000 | 0.0000 | 0.0000 | 0.0000 | 0.26 |
| 21 | 0.0000 | 0.0000 | 0.0000 | 0.0000 | 0.0000 | 0.0000 | 0.0000 | 0.83 |
| 22 | 0.0000 | 0.0000 | 0.0000 | 0.0000 | 0.0000 | 0.0000 | 0.0000 | 1.35 |
| 23 | 0.0000 | 0.0000 | 0.0000 | 0.0000 | 0.0000 | 0.0000 | 0.0000 | 2.47 |
| 24 | 0.0000 | 0.0000 | 0.0000 | 0.0000 | 0.0000 | 0.0000 | 0.0000 | 1.94 |
| 25 | 0.0000 | 0.0000 | 0.0000 | 0.0000 | 0.0000 | 0.0000 | 0.0000 | −1.1 |
| 26 | 0.0000 | 0.0000 | 0.0000 | 0.0000 | 0.0000 | 0.0000 | 0.0000 | 1.74 |
| 27 | 0.0000 | 0.0000 | 0.0000 | 0.0000 | 0.0000 | 0.0000 | 0.0000 | −1.08 |
| 28 | 0.0000 | 0.0000 | 0.0000 | 0.0000 | 0.0000 | 0.0000 | 0.0000 | −0.46 |
| 29 | 0.0000 | 0.0000 | 0.0160 | 0.0000 | 0.0000 | 0.0000 | 0.0000 | 2.38 |
| 30 | 0.0000 | 0.0000 | 0.0299 | 0.0000 | 0.0000 | 0.0000 | 0.0000 | 0.36 |

TABLE 7
Predicted and Experimental Values of Active Concentration (log1/C) of
Mitomycins 1-30 Against Human Tumor

| Compound | R | Prediction | Experimenter | resid. |
|---|---|---|---|---|
| 1 | NH2 | 7.711772 | 7.7 | −0.01177 |
| 2 | HOC3H6NH | 7.071587 | 6.98 | −0.09159 |
| 3 | HC═CCH2—NH | 8.102683 | 8.46 | 0.357317 |
| 4 | tetrahydrofuryl-NH | 7.245377 | 7.13 | −0.11538 |
| 5 | 2-furyl-C2H4—NH | 7.565948 | 7.34 | −0.22595 |
| 6 | 2-pyridyl-C2H4—NH | 7.38 | 7.38 | −1.3E−14 |
| 7 | C6H5NH | 8.862808 | 8.78 | −0.08281 |
| 8 | 4-H2N—C6H4—NH | 7.642204 | 7.83 | 0.187796 |
| 9 | 4-F—C6H4—NH | 8.67 | 8.67 | −2E−14 |
| 10 | 4-Br—C6H4—NH | 8.72 | 8.72 | 1.78E−14 |
| 11 | 3-I—C6H4—NH | 8.7268 | 8.9 | 0.1732 |
| 12 | 4-I—C6H4—NH | 8.771307 | 8.77 | −0.00131 |
| 13 | 4-OH—C6H4—NH | 7.965666 | 7.88 | −0.08567 |
| 14 | 4-NO2—C6H4—NH | 9.015853 | 9.07 | 0.054147 |
| 15 | 3-I-4-OH—C6H3—NH | 7.931492 | 7.76 | −0.17149 |
| 16 | 4-OH-3-NO2—C6H3—NH | 7.76895 | 7.71 | −0.05895 |
| 17 | 5-indolyl-NH | 8.75 | 8.75 | −8.9E−15 |
| 18 | 4-methyl-thiazolyl-NH | 8.679922 | 8.69 | 0.010078 |
| 19 | 3-pyrazolyl-NH | 7.388116 | 7.38 | −0.00812 |
| 20 | CH3O | 9.602933 | 9.52 | −0.08293 |
| 21 | c-C3H5—O | 9.080572 | 9.2 | 0.119428 |
| 22 | c-C3H5—CH2—O | 9.304672 | 9.43 | 0.125328 |
| 23 | c-C4H7—CH2—O | 9.787183 | 9.66 | −0.12718 |
| 24 | C6H5—CH2—O | 9.481265 | 9.21 | −0.27126 |
| 25 | HO—C2H4—O | 8.397708 | 8.31 | −0.08771 |
| 26 | C6H5—O—C2H4—O | 8.808812 | 9.48 | 0.671188 |
| 27 | HO—C2H4—O—C2H4—O | 7.88795 | 7.32 | −0.56795 |
| 28 | CH3—O—C2H4—O—C2H4—O | 7.786789 | 8.24 | 0.453211 |
| 29 | C6H5—S—C2H4—O | 9.480943 | 9.16 | −0.32094 |
| 30 | HO—C2H4—SS—C2H4—O | 8.490691 | 8.65 | 0.159309 |

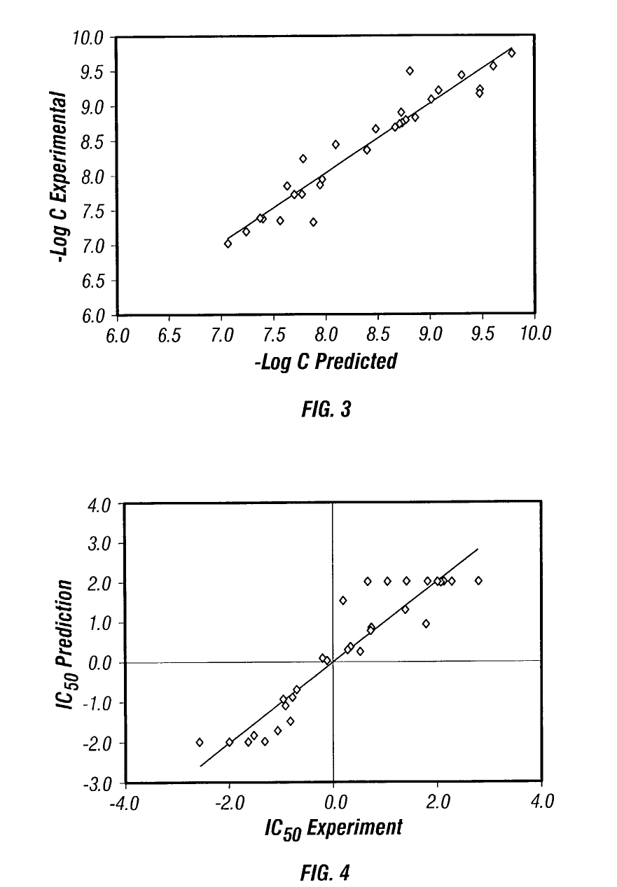

As can be seen in Table 7, above (presented graphically in FIG. 3), the modified 3D CAN(TM) allows us to quantify the set of bioactivity parameters of substituted mitomycins with accuracy, considerably higher then has been previously reported by other authors.

### Example 3

### Application of the Modified 3D CAN(TM) to Quantification of Inhibiting Dosage (IC₅₀) in Non Steroidal Anti-Inflammatory (NSAID)

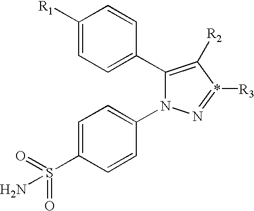

3D CAN(TM) has been applied to the series of compounds selected from the group of molecules known as NSAID. The common mechanism of action for all NSAIDs is the inhibition of the enzyme cyclooxgenase (COX). COX is necessary in the formation of prostaglandins. This enzyme actually has two known forms, COX-1 which protects the stomach lining and intestine, and COX-2 that is involved in making the prostaglandins that are important in the process of inflammation.

The corresponding IC₅₀ values (in mmol) have been processed within the standard 3D CAN(TM) schemata, where the parameters are modeled as the following:

$$log ( \frac{{IC}_{50}}{{IC}_{50}^{0}} ) = {\sum }_{i \neq rc}^{N - 1} \frac{{ic}_{i}}{{r}_{rc - i}^{2}}$$

where N is the number of atoms in the molecule,

r_{rc-l} is the distance between atom i and the reaction center (rc)

icᵢ is introduced operational atomic parameters, reflecting the ability of an atom of a certain type to contribute into overall IC₅₀−value. IC₅₀⁰ corresponds to unsubstituted compound (all R are hydrogen).

In order to obtain a simplified version of equation above (not taking into account the standard unsubstituted compound of a series) the experimental values IC₅₀ᵢ have been modeled in the form:

$$log {IC}_{50} = const + {\sum }_{i \neq rc}^{N - 1} \frac{{ic}_{i}}{{r}_{rc - i}^{2}}$$

Several common atoms have been tested as a potential reaction center of the series. The best solution was found when 3-C(aromatic) atom is considered to be a rc. This atom has been marked with a star in the structure above. Using this atom as rc, the operational atomic parameters have been established as the following for inhibition of COX 1 and COX 2 (Tables 8 and 9 respectively):

TABLE 8
Operational atomic parameters IC₅₀, derived for
the presented atomic types from IC₅₀ of
NSAIDs against COX1.

Const1

| | 1.68115 | 33.5947 |
|---|---|---|
| Atomic type | COX1 | +/− |
| H | −6.08934 | 1.8399 |
| C4 | 0.568013 | 0.995614 |
| C3 | 23.11429 | 40.81493 |
| C2 | −4.4601 | 6.046124 |
| Car | −0.80518 | 0.970352 |
| N1 | −11.0634 | 18.46849 |
| O2 | −7.97921 | 1.972359 |
| O1 | −70.3575 | 107.7834 |
| F | −12.2981 | 2.722617 |
| Cl | −26.2135 | 6.007283 |
| Br | −23.2818 | 7.692727 |
| S2 | −5.20356 | 12.24237 |
| S6 | 107.0076 | 174.6307 |
| O— | −27.0186 | 6.968644 |
| N2 | −10.5631 | 3.949839 |
| NO | 63.15832 | 141.5853 |

TABLE 9
Operational atomic parameters IC₅₀, derived for
al the presented atomic types from IC₅₀ of
NSAIDs against COX2.

Const2

| | 63.19161 | 47.7953 |
|---|---|---|
| Atomic type | COX2 | +/− |
| H | −2.1685 | 2.617633 |
| C4 | −3.7513 | 1.416463 |
| C3 | −75.8065 | 58.06755 |
| C2 | 0.6020 | 8.601842 |
| Car | 0.3616 | 1.380523 |
| N1 | −2.9799 | 26.27519 |
| O2 | 0.9039 | 2.806083 |
| O1 | 205.8184 | 153.3439 |
| F | −0.7661 | 3.873477 |
| Cl | −11.5938 | 8.546583 |
| Br | −21.9553 | 10.94447 |
| S2 | −3.3968 | 17.41726 |
| S6 | −328.899 | 248.4477 |
| O— | −15.8344 | 9.914316 |
| N2 | −3.4229 | 5.619451 |
| NO | −284.421 | 201.434 |

The IC₅₀ has been modeled in form of the following correlations (the statistical parameters are present)

$log {IC}_{50}^{COX1} = {const}_{1} + {\sum }_{i \neq rc}^{N - 1} \frac{{cox1}_{i}}{{r}_{rc - i}^{2}}$
Regression Statistics

| | |
|---|---|
| Multiple R | 0.938227 |
| R Square | 0.880269 |
| Adjusted R Square | 0.743434 |
| Standard Error | 0.778754 |
| Observations | 31 |

$log {IC}_{50}^{COX2} = {const}_{2} + {\sum }_{i \neq rc}^{N - 1} \frac{{cox2}_{i}}{{r}_{rc - i}^{2}}$
Regression Statistics

| | |
|---|---|
| Multiple R | 0.8469 |
| R Square | 0.717239 |
| Adjusted R | 0.394084 |
| Square | |
| Standard Error | 1.107937 |
| Observations | 31 |

Thus, the applied approach allowed a reasonably accurate quantitative interpretation of bioactivity of considered drugs against COX1 and COX2. The values of the estimated atomic operational contributions ic in the above equations can be used for prediction of unknown values of IC₅₀ for compounds, constituted from the atom types presented in Tables 10 and 11.

TABLE 10
Predicted vs. experimental IC₅₀ of NSAIDs against COXI

| Nr | R1 | ] | R3 | IC₅₀ pred | IC₅₀ exper | resid. |
|---|---|---|---|---|---|---|
| 1 | H | ] | CHF2 | 0.194 | 1.528 | 1.334 |
| 2 | H | ] | CH2F | 1.043 | 2.000 | 0.957 |
| 3 | F | ] | H | 1.812 | 2.000 | 0.188 |
| 4 | Cl | ] | CH2OH | 1.414 | 2.000 | 0.586 |
| 5 | Cl | ] | CH2CN | 2.789 | 2.000 | −0.789 |
| 6 | Cl | ] | C6H4—OCH3(4) | 1.783 | 0.929 | −0.854 |
| 7 | Cl | ] | C6H4-2-SH-5-Cl | 2.119 | 2.000 | −0.119 |
| 8 | F | ] | CN | 0.660 | 2.000 | 1.340 |
| 9 | F | ] | COOH | 2.278 | 2.000 | −0.278 |
| 10 | F | ] | COOCH3 | 2.000 | 2.000 | 0.000 |
| 11 | F | ] | CONH2 | 2.005 | 2.000 | −0.005 |
| 12 | F | ] | CONHC6H4—Cl (4) | 0.283 | 0.283 | 0.000 |
| 13 | H | ] | OCH3 | 2.059 | 2.000 | −0.059 |
| 14 | Cl | ( | CF3 | 1.444 | 1.187 | −0.257 |
| 15 | H | ] | CF3 | −0.220 | 0.081 | 0.301 |
| 16 | Cl | ( | CF3 | −0.115 | 0.032 | 0.146 |
| 17 | H | ( | CF3 | −2.586 | −2.000 | 0.586 |
| 18 | H | ( | H | −0.833 | −1.491 | −0.659 |
| 19 | Cl | ] | H | −0.760 | −0.940 | −0.180 |
| 20 | H | ] | H | −1.475 | −1.752 | −0.277 |
| 21 | H | ( | H | −1.645 | −2.000 | −0.355 |
| 22 | CH3 | ( | H | −1.330 | −2.000 | −0.670 |
| 23 | H | ] | H | −0.910 | −1.086 | −0.176 |
| 24 | Cl | ] | H | −1.076 | −1.716 | −0.640 |
| 25 | H | ] | H | −0.708 | −0.708 | 0.000 |
| 26 | H | ( | CH3 | 0.513 | 0.237 | −0.277 |
| 27 | Cl | ( | CH2OH | 0.731 | 0.770 | 0.039 |
| 28 | Cl | ( | CN | 0.733 | 0.854 | 0.121 |
| 29 | Cl | ( | COOH | −2.000 | −2.000 | 0.000 |
| 30 | Cl | ( | COOCH3 | 0.384 | 0.387 | 0.004 |
| 31 | Cl | ( | CONH2 | −0.938 | −0.944 | −0.007 |

TABLE 11
Predicted vs. experimental IC₅₀ of NSAIDs against COX2;

| Nr | R1 | ] | R3 | IC₅₀ pred | IC₅₀ exper | resid. |
|---|---|---|---|---|---|---|
| 1 | H | ] | CHF2 | 0.697 | −0.886 | −1.583 |
| 2 | H | ] | CH2F | 0.060 | −0.699 | −0.759 |
| 3 | F | ] | H | −0.029 | 2.000 | 2.029 |
| 4 | Cl | ] | CH2OH | 0.200 | −0.081 | −0.281 |
| 5 | Cl | ] | CH2CN | −0.716 | −0.921 | −0.205 |
| 6 | Cl | ] | C6H4—OCH3(4) | −0.379 | −1.000 | −0.621 |
| 7 | Cl | ] | C6H4-2-SH-5-Cl | −0.481 | −1.284 | −0.803 |
| 8 | F | ] | CN | −0.950 | −0.469 | 0.482 |
| 9 | F | ] | COOH | 2.005 | 2.000 | −0.005 |
| 10 | F | ] | COOCH3 | 2.000 | 2.000 | 0.000 |
| 11 | F | ] | CONH2 | 2.034 | 2.000 | −0.034 |
| 12 | F | ] | CONHC6H4—Cl (4) | −1.252 | −1.252 | 0.000 |
| 13 | H | ] | OCH3 | 2.581 | 2.000 | −0.581 |
| 14 | Cl | ( | CF3 | 1.355 | 2.276 | 0.921 |
| 15 | H | ] | CF3 | 3.097 | 2.770 | −0.328 |
| 16 | Cl | ( | CF3 | 1.415 | 1.658 | 0.242 |
| 17 | H | ( | CF3 | 0.209 | 1.097 | 0.888 |
| 18 | H | ( | H | 1.465 | 1.310 | −0.155 |
| 19 | Cl | ] | H | −0.052 | 1.509 | 1.561 |
| 20 | H | ] | H | −0.575 | −0.668 | −0.093 |
| 21 | H | ( | H | −0.746 | −1.673 | −0.927 |
| 22 | CH3 | ( | H | 0.561 | 1.119 | 0.558 |
| 23 | H | ] | H | 1.114 | 0.538 | −0.576 |
| 24 | Cl | ] | H | −0.330 | −1.297 | −0.966 |
| 25 | H | ] | H | −1.473 | −1.473 | 0.000 |
| 26 | H | ( | CH3 | 0.815 | 1.553 | 0.737 |
| 27 | Cl | ( | CH2OH | 0.235 | 0.469 | 0.234 |
| 28 | Cl | ( | CN | 1.187 | 2.000 | 0.813 |
| 29 | Cl | ( | COOH | −1.845 | −1.845 | 0.000 |
| 30 | CI | ( | COOCH3 | 0.800 | 0.796 | −0.004 |
| 31 | Cl | ( | CONH2 | 0.506 | −0.037 | −0.543 |

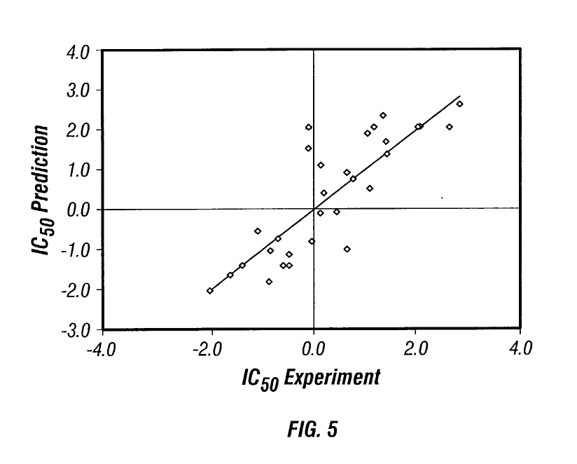

The estimated 3D CAN(TM) correlations are graphically presented on FIGS. 4 and 5 respectively.

The examples and embodiments described in this patent are for illustrative purposes only and various modifications or changes will be suggested to persons skilled in the art and are to be included within the disclosure in this application and scope of the claims. All publications, patents and patent applications cited in this patent are hereby incorporated by reference in their entirety for all purposes to the same extent as if each individual publication, patent or patent application were specifically and individually indicated to be so incorporated by reference.

1. A method for calculating a biological characteristic property of a molecule, where the molecule comprises one or more substituent parts, the method comprising the steps of
selecting one or more contributing substituent parts;
for each contributing substituent part, calculating a distance from the substituent part to a reaction center;
for each contributing substituent part, calculating the contribution of the substituent part to the biological characteristic property of the molecule, where the contribution is equal to a function of the distance of the substituent part to the reaction center multiplied by a weight factor for the substituent part, and where the function has a functional form that is substantially the same for all substituent parts; and
calculating the biological characteristic property of the molecule by summing the contributions from the contributing substituent parts of the molecule.

2. The method of claim 1, wherein the biological characteristic property is selected from the group consisting of therapeutic index, effective dosage, inhibiting concentration, lethal dosage, hydrophobicity, solubility, toxicity, brain blood barrier crossing concentration, kinetics of biotransformation pathways, rate constant for in vivo or in vitro oxidation, rate constant for in vivo or in vitro phosphorylation, rate constant for in vivo or in vitro alkylation, and rate constant for in vivo or in vitro glycosylation, absorption, clearance, metabolic stability, pharmacokinetics, t_{½} biological reactivity, bioefficacy, and binding affinity.

3. The method of claim 2, wherein the biological characteristic property is the therapeutic index, bioefficacy, toxicity, or binding affinity.

4. The method of claim 2 wherein the effective dosage is ED₅₀, ED₃₀, or ED₈₀.

5. The method of claim 2 wherein the inhibiting dosage is IC₅₀.

6. The method of claim 2, wherein the lethal dosage is LD₁₀₀, or LD₅₀.

7. The method of claim 1, wherein the biological characteristic property is a property that is characteristic of the interaction of the molecule with a subject organism or the effect of the molecule on a subject organism.

8. The method of claim 7, wherein the subject organism is an animal or a plant

9. The method of claim 7, wherein the subject organism is an animal.

10. The method of claim 9, wherein the animal is a mammal.

11. The method of claim 10, wherein the mammal is selected from the group consisting of mouse, guinea pig, rabbit, frog, dog and rat.

12. The method of claim 10, wherein the mammal is a human.

13. The method of claim 8, wherein the plant is selected from the group consisting of soybean, corn, rice, wheat, canola, and potato.

14. The method of claim 7, wherein the subject organism is a microorganisms.

15. The method of claim 14, wherein the microorganisms is selected from the group consisting of bacteria, algae, archae and yeast.

16. The method of claim 7, wherein the subject organism is a fungi.

17. The method of claim 7, wherein the subject organism is a virus.

18. The method of claim 1, wherein the biological characteristic property is a property characteristic of the interaction of the molecule with or the effect of the molecule on cells, tissues, organelles or organs of an organism.

19. The method of claim 1, wherein the molecule is an aniline mustard, an NSAID, or a Mitomycin.

20. The method of claim 1, wherein the molecule is selected from the group consisting of organic molecules, inorganic molecules, neutral molecules, radicals, anions, cations, ionic salts, metallo-organic compounds and coordination compounds.

21. The method of claim 1, wherein a substituent part of the molecule is an atom contained in the molecule or a group of connected atoms contained in the molecule.

22. The method of claim 1, wherein the contributing substituent parts include all substituent parts of the molecule except one.

23. The method of claim 1, wherein the reaction center is a point in space.

24. The method of claim 23, wherein the point is space is an atom contained in the molecule.

25. The method of claim 1, wherein the reaction center comprises a substituent part of the molecule.

26. The method of claim 1, wherein the reaction center is one of the substituent parts of the molecule.

27. The method of claim 26, wherein the contributing substituent parts include all substituent parts in the molecule except the reaction center substituent part.

28. The method of claim 1, wherein the function of the distance is of the form of an inverse function of the distance.

29. The method of claim 28 wherein the function of the distance goes as the inverse of the square of the distance.

30. The method of claim 28, wherein the function of the distance goes as the inverse of the cube of the distance.

31. The method of claim 28, wherein the function of the distance goes as sum of the inverse of the square of the distance and the inverse of the cube of the distance.

32. The method of claim 1, wherein the weight factor is calculated as a regression coefficient for a multivariate regression analysis calculated for a series of molecules.

33. The method of claim 32, wherein for the multivariate regression analysis a dependent variable is the biological characteristic property for one of molecules in the series and there is an independent variable for each type of substituent part present in the series of molecules, and for a particular independent variable the value of the dependent variable corresponding to a particular substituent part is equal to a sum over all of the particular substituent parts in the molecule corresponding to the independent variable of the function of the distance from the reaction center to the particular substituent part.

34. The method of claim 32, wherein the series of molecules comprise analogs of the molecule.

35. The method of claim 32, wherein the series of molecules comprise molecules that have the same reaction center as the molecule.

36. The method of claim 32, wherein the reaction center is a point in space or a substituent part of the molecule and the reaction center is selected by a method comprising
for a first reaction center, performing the multivariable regression analysis and determining characteristic of the multivariable regression analysis,
for a second reaction center, performing the multivariable regression analysis and determining a second characteristic of the multivariable regression analysis,
identifying the reaction center as that reaction center with the multivariable regression analysis characteristic satisfying a predetermined criteria.

37. The method of claim 36, wherein the characteristic of the multivariable regression analysis is the global regression coefficient calculated for the multivariable regression and the predetermined criteria selects from the reaction center with the highest global regression coefficient.

38. The method of claim 36, wherein the characteristic of the multivariable regression analysis is the global standard error of the multivariable regression and the predetermined criteria selects from the reaction center with the lowest global standard error.

39. The method of claim 1, wherein the molecule has one or more measured properties and wherein the biological characteristic property of the molecule is calculated by summing the contributions from the contributing substituent parts of the molecule plus a contribution comprising a measured property of the molecule multiplied by a weight factor.

40. The method of claim 39, wherein one of the measured properties of the molecule is the hydrophobicity of the molecule.

41. The method of claim 39, wherein the measured property weight factor is calculated as a regression coefficient for a multivariate regression analysis calculated for a series of molecules.

42. A method for calculating a biological characteristic property of a molecule, where the molecule comprises one or more substituent parts, the method comprising the steps of
selecting one of the substituent parts as a reaction center;
for each substituent part other than the reaction center, calculating a distance from the substituent part to the reaction center;
for each substituent part other than the reaction center, calculating the contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule;
calculating the biological characteristic property of the molecule by summing the contributions from the contributing substituent parts of the molecule.

43. The method of claim 42, wherein the biological characteristic property is selected from the group consisting of therapeutic index, IC₅₀, ED₅₀, LD₅₀, hydrophobicity, solubility, toxicity, brain blood barrier crossing concentration, kinetics of biotransformation pathways, rate constant for in vivo or in vitro oxidation, rate constant for in vivo or in vitro phosphorylation, rate constant for in vivo or in vitro alkylation, and rate constant for in vivo or in vitro glycosylation.

44. The method of claim 42, wherein the biological characteristic property is the therapeutic index.

45. The method of claim 42, wherein the biological characteristic property is a property characteristic of the interaction of the molecule with or the effect of the molecule on a subject organism.

46. The method of claim 45, wherein the subject organism is an animal or a plant.

47. The method of claim 45, wherein the subject organism is an animal.

48. The method of claim 47, wherein the animal is a human.

49. The method of claim 42, wherein the molecule is an aniline mustard, an NSAID, or a Mitomycin.

50. The method of claim 42, wherein the molecule is selected from the group consisting of organic molecules, inorganic molecules, neutral molecules, radicals, anions, cations, ionic salts, metallo-organic compounds and coordination compounds.

51. The method of claim 42, wherein substituent part of the molecule is an atom contained in the molecule or a group of connected atoms contained in the molecule.

52. The method of claim 42, wherein for the multivariate regression analysis a dependent variable is the biological characteristic property for one of molecules in the series and there is an independent variable for each type of substituent part present in the series of molecules, and for a particular independent variable the value of the dependent variable corresponding a particular substituent part is equal to a sum over all of the particular substituent parts in the molecule corresponding to the independent variable of the inverse square of the distance from the reaction center to the particular substituent part.

53. The method of claim 42, wherein the reaction center is selected by a method comprising
for a first reaction center, performing the multivariable regression analysis and determining a first characteristic of the multivariable regression analysis,
for a second reaction center, performing the multivariable regression analysis and determining a second characteristic of the multivariable regression analysis,
identifying the reaction center as that reaction center with the multivariable regression analysis characteristic satisfying a predetermined criteria.

54. The method of claim 53 wherein the characteristic of the multivariable regression analysis is the global regression coefficient and the predetermined criteria selects for the reaction center with the highest global regression coefficient.

55. The method of claim 53 wherein the characteristic of the multivariable regression analysis is the global standard error and the predetermined criteria selects for the reaction center with the lowest standard error.

56. The method of claim 42, wherein the molecule has one or more measured properties and wherein the biological characteristic property of the molecule is calculated by summing the contributions from the contributing substituent parts of the molecule and a contribution comprising a measured property of the molecule multiplied by a weight factor.

57. The method of claim 56, wherein one of the measured properties of the molecule is the hydrophobicity of the molecule.

58. The method of claim 42, wherein the measured property weight factor is calculated as a regression coefficient for a multivariate regression analysis calculated for the series of molecules.

59. A method for calculating a biological characteristic property of a molecule, where the molecule has a hydrophobicity and the molecule comprises one or more substituent parts and the substituent parts are atoms contained in the molecule or groups of connected atoms contained in the molecule, the method comprising
selecting one of the substituent parts as a reaction center;
for each substituent part other than the reaction center, calculating the distance from the substituent part to the reaction center;
for each substituent part other than the reaction center, calculating a contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule;
calculating the contribution of the hydrophobicity as equal to the value of the hydrophobicity multiplied by a weight factor calculated as a regression coefficient for a multivariate regression analysis calculated for a series of molecules comprising analogs of the molecule; and
calculating the biological characteristic property of the molecule by summing the contributions from the contributing substituent parts of the molecule and the contribution from the hydrophobicity.

60. The method of claim 59, wherein the biological characteristic property is selected from the group consisting of therapeutic index, inhibiting concentration, effective dosage, lethal dosage, hydrophobicity, solubility, toxicity, brain blood barrier crossing concentration, kinetics of biotransformation pathways, rate constant for in vivo or in vitro oxidation, rate constant for in vivo or in vitro phosphorylation, rate constant for in vivo or in vitro alkylation, and rate constant for in vivo or in vitro glycosylation, absorption, clearance, metabolic stability, pharmacokinetics, t_{½} biological reactivity, bioefficacy, and binding affinity.

61. The method of claim 59, wherein the biological characteristic property is the therapeutic index, bioefficacy, toxicity, or binding affinity.

62. The method of claim 60 wherein the effective dosage is ED₅₀, ED₃₀, or ED₈₀.

63. The method of claim 60 wherein the inhibiting dosage is IC₅₀.

64. The method of claim 60 wherein the lethal dosage is LD₁₀₀, or LD₅₀.

65. The method of claim 59, wherein the biological characteristic property is a property that is characteristic of the interaction of the molecule with a subject organism or the effect of the molecule on a subject organism.

66. The method of claim 65, wherein the subject organism is an animal or a plant.

67. The method of claim 65, wherein the subject organism is an animal.

68. The method of claim 67, wherein the animal is a human.

69. The method of claim 59, wherein the molecule is an aniline mustard, an NSAID, or a Mitomycin.

70. The method of claim 59, wherein the molecule is selected from the group consisting of organic molecules, inorganic molecules, neutral molecules, radicals, anions, cations, ionic salts, metallo-organic compounds and coordination compounds.

71. The method of claim 59, wherein for the multivariate regression analysis a dependent variable is the biological characteristic property for one of molecules in the series and there is an independent variable for each type of substituent part present in the series of molecules, and for a particular independent variable the value of the dependent variable corresponding a particular substituent part is equal to a sum over all of the particular substituent parts in the molecule corresponding to the independent variable of the inverse square of the distance from the reaction center to the particular substituent part.

72. The method of claim 59, wherein the reaction center is identified by a method comprising the steps of
for a first reaction center, performing the multivariable regression analysis and determining a first characteristic of the multivariable regression analysis,
for a second reaction center, performing the multivariable regression analysis and determining a second characteristic of the multivariable regression analysis,
identifying the reaction center as that reaction center with the multivariable regression analysis characteristic satisfying a predetermined criteria.

73. The method of claim 72, wherein the characteristic of the multivariable regression analysis is the global regression coefficient and the predetermined criteria selects for the reaction center with the highest global regression coefficient

74. The method of claim 72, wherein the characteristic of the multivariable regression analysis is the global standard error and the predetermined criteria selects for the reaction center with the lowest global standard error.

75. A system for calculating a biological characteristic property of a molecule, where the molecule comprises one or more substituent parts, the system comprising:
a processor; and
a computer readable medium having computer readable program code means embodied therein for causing the system to calculate a biological characteristic property of a molecule, the computer readable program code means comprising: (1) a computer readable program code means for causing a computer to carry out the step of selecting one or more contributing substituent parts; (2) a computer readable program code means for causing a computer to carry out the step of, for each contributing substituent part, calculating a distance from the substituent part to a reaction center; (3) a computer readable program code means for causing a computer to carry out the step of, for each contributing substituent part, calculating the contribution of the substituent part to the biological characteristic property of the molecule, where the contribution is equal to a function of the distance of the substituent part to the reaction center multiplied by a weight factor for the substituent part, and where the function has a functional form that is substantially the same for all substituent parts; and (4) a computer readable program code means for causing a computer to carry out the step of calculating the biological characteristic property of the molecule by summing the contributions from the contributing substituent parts of the molecule.

76. A system for calculating a biological characteristic property of a molecule, where the molecule comprises one or more substituent parts, the system comprising:
a processor; and
a computer readable medium having computer readable program code means embodied therein for causing the system to calculate a biological characteristic property of a molecule, the computer readable program code means comprising: (1) a computer readable program code means for causing a computer to carry out the step of selecting one of the substituent parts as a reaction center; (2) a computer readable program code means for causing a computer to carry out the step of, for each substituent part other than the reaction center, calculating a distance from the substituent part to the reaction center; (3) a computer readable program code means for causing a computer to carry out the step of, for each substituent part other than the reaction center, calculating the contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule; and (4) a computer readable program code means for causing a computer to carry out the step of calculating the biological characteristic property of the molecule by summing the contributions from the contributing substituent parts of the molecule.

77. A system for calculating a biological characteristic property of a molecule, where the molecule comprises one or more substituent parts, the system comprising:
a processor; and
a computer readable medium having computer readable program code means embodied therein for causing the system to calculate a biological characteristic property of a molecule, the computer readable program code means comprising: (1) a computer readable program code means for causing a computer to carry out the step of selecting one of the substituent parts as a reaction center; (2) a computer readable program code means for causing a computer to carry out the step of, for each substituent part other than the reaction center, calculating the distance from the substituent part to the reaction center; (3) a computer readable program code means for causing a computer to carry out the step of, for each substituent part other than the reaction center, calculating a contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule; (4) a computer readable program code means for causing a computer to carry out the step of calculating the contribution of the hydrophobicity as equal to the value of the hydrophobicity multiplied by a weight factor calculated as a regression coefficient for a multivariate regression analysis calculated for a series of molecules comprising analogs of the molecule; and (5) a computer readable program code means for causing a computer to carry out the step of calculating the biological characteristic property of the molecule by summing the contributions from the contributing substituent parts of the molecule and the contribution from the hydrophobicity.

78. An article of manufacture comprising a computer useable medium having computer readable program code means embodied therein for causing a computer to calculate a biological characteristic property of a molecule, where the molecule comprises one or more substituent parts, the computer readable program code means comprising: (1) a computer readable program code means for causing a computer to carry out the step of selecting one or more contributing substituent parts; (2) a computer readable program code means for causing a computer to carry out the step of, for each contributing substituent part, calculating a distance from the substituent part to a reaction center; (3) a computer readable program code means for causing a computer to carry out the step of, for each contributing substituent part, calculating the contribution of the substituent part to the biological characteristic property of the molecule, where the contribution is equal to a function of the distance of the substituent part to the reaction center multiplied by a weight factor for the substituent part, and where the function has a functional form that is substantially the same for all substituent parts; and (4) a computer readable program code means for causing a computer to carry out the step of calculating the biological characteristic property of the molecule by summing the contributions from the contributing substituent parts of the molecule.

79. An article of manufacture comprising a computer useable medium having computer readable program code means embodied therein for causing a computer to calculate a biological characteristic property of a molecule, where the molecule comprises one or more substituent parts, the computer readable program code means comprising: (1) a computer readable program code means for causing a computer to carry out the step of selecting one of the substituent parts as a reaction center; (2) a computer readable program code means for causing a computer to carry out the step of, for each substituent part other than the reaction center, calculating a distance from the substituent part to the reaction center; (3) a computer readable program code means for causing a computer to carry out the step of, for each substituent part other than the reaction center, calculating the contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule; and (4) a computer readable program code means for causing a computer to carry out the step of calculating the biological characteristic property of the molecule by summing the contributions from the contributing substituent parts of the molecule.

80. An article of manufacture comprising a computer useable medium having computer readable program code means embodied therein for causing a computer to calculate a biological characteristic property of a molecule, where the molecule comprises one or more substituent parts, the computer readable program code means comprising: (1) a computer readable program code means for causing a computer to carry out the step of selecting one of the substituent parts as a reaction center; (2) a computer readable program code means for causing a computer to carry out the step of, for each substituent part other than the reaction center, calculating the distance from the substituent part to the reaction center; (3) a computer readable program code means for causing a computer to carry out the step of, for each substituent part other than the reaction center, calculating a contribution of the substituent part to the biological characteristic property of the molecule, where the contribution is equal to the inverse of the square of the distance of the substituent part to the reaction center multiplied by a weight factor for the substituent part, and where the weight factor is calculated as a regression coefficient for a multivariate regression analysis calculated for a series of molecules comprising analogs of the molecule; (4) a computer readable program code means for causing a computer to carry out the step of calculating the contribution of the hydrophobicity as equal to the value of the hydrophobicity multiplied by a weight factor calculated as a regression coefficient for a multivariate regression analysis calculated for a series of molecules comprising analogs of the molecule; and (5) a computer readable program code means for causing a computer to carry out the step of calculating the biological characteristic property of the molecule by summing the contributions from the contributing substituent parts of the molecule and the contribution from the hydrophobicity.

81. A molecule comprising one or more substituent parts chosen to affect a biological characteristic property of the molecule, where the effect of the one or more substituent parts is calculated by the method according to claim 1.

82. A molecule comprising one or more substituent parts chosen to affect a biological characteristic property of the molecule, where the effect of the one or more substituent parts is calculated by the method according to claim 42.

83. A molecule comprising one or more substituent parts chosen to affect a biological characteristic property of the molecule, where the effect of the one or more substituent parts is calculated by the method according to claim 59.

84. A molecule synthesized after determining a likely biological characteristic property of the molecule, where the effect of the biological characteristic property of the molecule is calculated by the method according to claim 1.

85. A molecule synthesized after determining a likely biological characteristic property of the molecule, where the effect of the biological characteristic property of the molecule is calculated by the method according to claim 42.

86. A molecule synthesized after determining a likely biological characteristic property of the molecule, where the effect of the biological characteristic property of the molecule is calculated by the method according to claim 59.
